(19) United States
(12) Patent Application Publication
(10) Pub. No.: US 2024/0335488 A1
(43) Pub. Date: Oct. 10, 2024

(54) NOVEL LACTOBACILLUS HELVETICUS UA881 STRAIN AND PROBIOTIC COMPOSITION FOR IMPROVING INTESTINAL PERMEABILITY AND METABOLIC DISORDERS

(71) Applicant: NUTRAREX BIOTECH CO., LTD., Taichung City (TW)

(72) Inventors: Meei-Yn LIN, Taichung City (TW); Pin-Chao HUANG, Taichung City (TW); Shao Yu LEE, Taichung City (TW); Jyun-Ting SYU, Taichung City (TW); Chin-Hsiu YU, Taichung City (TW)

(21) Appl. No.: 18/626,672

(22) Filed: Apr. 4, 2024

Related U.S. Application Data
(60) Provisional application No. 63/457,812, filed on Apr. 7, 2023.
(60) Provisional application No. 63/463,317, filed on May 2, 2023.

(51) Int. Cl. A61K 35/747; A61P 1/04; A61P 3/02; A61P 3/06; A61P 19/06; A61P 29/00; A61P 39/06; C12N 1/20; C12R 1/225

(52) U.S. Cl. CPC A61K 35/747; A61P 1/04; A61P 3/02; A61P 3/06; A61P 19/06; A61P 29/00; A61P 39/06; C12N 1/20; C12R 2001/225

(57) ABSTRACT

The present disclosure provides a novel Lactobacillus helveticus UA881 strain and/or its probiotic composition, as well as their uses for improving intestinal permeability and metabolic disorders. These include lowering levels of triacylglyceride and cholesterol levels, improving leaky gut and metabolism disorders, degrading uric acid and purine nucleosides, relieving gout arthritis, activating antioxidant systems, and generating micro-nutrients. The novel Lactobacillus helveticus UA881 strain and/or its metabolites can be used to prepare medicaments, food products, health food, and external products for these purposes. The invention includes the Lactobacillus helveticus UA881 strain, deposited at the National Institute of Technology and Evaluation (NITE) under accession number NITE BP-03802.

### CROSS-REFERENCE TO RELATED APPLICATION

This application claims priorities of Provisional application No. 63/457,812, filed on Apr. 7, 2023, and Provisional application No. 63/463,317, filed on May 2, 2023, the content of which is incorporated herein in its entirety by reference.

### BACKGROUND OF THE INVENTION

### 1. Field of the Invention

The present invention relates to a novel Lactobacillus helveticus UA881 strain and the probiotic composition and uses thereof. In particular, the present invention relates to a novel Lactobacillus helveticus UA881 strain which improves intestinal permeability, alleviates hyperlipidemia, hypercholesterolemia and hyperuricemia, relieves intestinal inflammation, activates antioxidant systems, and generates micro-nutrients.

### 2. The Prior Art

The intestine is an important digestive organ of the human body. 70% of immune cells exist in the intestine, which can initiate immune responses to resist the invasion of pathogens. It also secretes a variety of hormones to regulate physiological functions and participate in energy metabolism. The intestine can form a protective barrier. The single layer of intestinal epithelial cells forms a complete defense barrier to isolate the invasion of bacteria, toxins, etc. When the intestinal mucosa is damaged, the production, synthesis and absorption of nutrient sources in the body are hindered, eventually leading to inflammation, metabolic abnormalities, three highs and other diseases.

The intestinal mucosa provides a selectively permeable barrier for nutrient absorption and protection from external factors. It is composed of epithelial cells, immune cells and their secretions. Gut microbiota participates in regulating intestinal barrier integrity and function to achieve homeostatic balance. Pathogens, xenobiotics, and foods can disrupt the intestinal barrier and promote systemic inflammation and tissue damage.

Following barrier injury, wound healing occurs in three phases: villus retraction, cell migration, and paracellular space closure. Cell migration, where cells extend forward and cover exposed surfaces, was assessed using a scratch injury model. Wound healing is completed by resealing tight junctions after restoration through cell migration, and analyzed by expression of zonula occludens-1 (ZO-1). Damage in the intestinal barrier causes increased permeability.

Cardiovascular disease is the silent killer of humans, and excessive triacylglyceride, cholesterol, and LDL are major risk factors. Heart disease and stroke are 1^{st} and 2^{nd} leading causes of death, respectively. There is a 400% higher incidence of heart disease and stroke in an excessive cholesterol population. A 10% reduction in LDL reduces the risk of strokes by 15.6%. There are 48% of global people with hyperlipidemia and 86 million US adults with excessive cholesterol. There is US $22.2 billion market size of hyperlipidemia drugs.

Red yeast rice is an effective dietary supplement to control blood lipid and cholesterol. However, the functional compound, monacolin K, belongs to the statin family. The US FDA warns that red yeast rice shares the same side effects with statin drugs, causing risks of muscle pain and rhabdomyolysis. Therefore, red yeast rice is forbidden to be marketed as a dietary supplement in the United States. Moreover, the US NIH also warns that some red yeast rice products contain a contaminant called citrinin, which is toxic and can damage the kidneys.

Probiotics may be a potent treatment for hyperlipidemia. It is a future avenue in promoting cardiovascular health. A few probiotic strains have the potential to relieve hyperlipidemia, majorly due to the degradation of lipids, cholesterol, and bile acids in the intestine. However, the ability is strongly strain-dependent.

A comprehensive solution for cardiovascular health is still lacking in nutraceutical ingredients or products. A single function cannot solve a complicated issue. Several probiotic strains on the market are claimed to have the function of improving cardiovascular health, due to their cholesterol or triacylglyceride reduction abilities. However, cardiovascular health is a complicated issue involving blood lipid profile, inflammation, oxidative stress, and micro-nutrients. Modulating cholesterol and triacylglyceride may not be enough to improve the overall condition.

At present, clinical drug treatments for leaky gut syndrome, metabolic disorders and gout arthritis have limited effects and serious side effects, and many patients cannot continue to be treated. More importantly, the drug only alleviates the symptoms, but fails to fundamentally solve the problem, so how to develop a new drug that can effectively relieve leaky gut syndrome, metabolic disorders and gout arthritis is an important issue that the present invention intends to solve here.

In order to solve the above-mentioned problems, those skilled in the art urgently need to develop a novel pharmaceutical composition for relieving leaky gut syndrome, metabolic disorders and gout arthritis for the benefit of a large group of people in need thereof.

### SUMMARY OF THE INVENTION

In order to solve the foregoing problems, an objective of the present invention is to provide a Lactobacillus helveticus UA881 strain, deposited in National Institute Technology and Evaluation (NITE) under an accession number NITE BP-03802.

Another objective of the present invention is to provide a probiotic composition, comprising a Lactobacillus helveticus UA881 strain and/or its metabolites, wherein the Lactobacillus helveticus UA881 strain is deposited in National Institute Technology and Evaluation (NITE) under an accession number NITE BP-03802.

According to an embodiment of the present invention, the Lactobacillus helveticus UA881 strain is viable, inactive or its metabolites.

Another objective of the present invention is to provide a method for producing folic acid in human intestine, lowering uric acid, purine nucleosides, cholesterol, and triacylglyceride by using the aforementioned probiotic composition.

According to an embodiment of the present invention, the probiotic composition is a medicament, a nutritional supplement, a health food, a food product, a skin health product, an external product, or a combination thereof.

Another objective of the present invention is to provide a method for relieving leaky gut syndrome, comprising administering to a subject in need thereof a pharmaceutical composition comprising an effective amount of the aforementioned probiotic composition.

According to an embodiment of the present invention, the probiotic composition increases trans-epithelial electrical resistance in colon adenocarcinoma cells.

According to an embodiment of the present invention, the probiotic composition promotes barrier repair on lipopolysaccharide (LPS)-induced delay in wound healing.

According to an embodiment of the present invention, the probiotic composition increases expression of zonula occludens-1 (ZO-1) protein and regulates expression of tight junction proteins in intestinal epithelial cells, thereby protecting intestinal barrier.

Another objective of the present invention is to provide a method for relieving metabolic disorders, comprising administering to a subject in need thereof a pharmaceutical composition comprising an effective amount of the aforementioned probiotic composition.

According to an embodiment of the present invention, the probiotic composition enhances macrophage glutathione (GSH) content.

According to an embodiment of the present invention, the probiotic composition has ability to degrade purine nucleosides.

According to an embodiment of the present invention, the probiotic composition has ability to degrade uric acid.

According to an embodiment of the present invention, the probiotic composition decreases serum triacylglyceride (TG).

According to an embodiment of the present invention, the probiotic composition reduces serum cholesterol levels to within the normal range.

According to an embodiment of the present invention, the probiotic composition reduces low-density lipoprotein (LDL) and increases high-density lipoprotein (HDL) in serum.

According to an embodiment of the present invention, the probiotic composition reduces pro-inflammatory cytokine secretion.

According to an embodiment of the present invention, the probiotic composition activates antioxidant systems and prevents oxidative damage.

According to an embodiment of the present invention, the probiotic composition enhances the level of vitamin B12 in serum.

According to an embodiment of the present invention, the probiotic composition reduces serum uric acid.

Another objective of the present invention is to provide a method for relieving gout arthritis, comprising administering to a subject in need thereof a pharmaceutical composition comprising an effective amount of the aforementioned probiotic composition.

According to an embodiment of the present invention, the pharmaceutical composition is a medicament, a nutritional supplement, a health food, a food product, a skin health product, an external product, or a combination thereof.

In summary, the Lactobacillus helveticus UA881 strain of the present invention has the effects on producing folic acid in the human intestine, degrading uric acid, purine nucleosides, cholesterol and triacylglyceride, relieving leaky gut syndrome and metabolic disorders through the experiments illustrated in the following examples.

### BRIEF DESCRIPTION OF THE DRAWINGS

The following drawings form part of the present specification and are included here to further demonstrate some aspects of the present invention, which can be better understood by reference to one or more of these drawings, in combination with the detailed description of the embodiments presented herein.

FIG. 1 shows that the Lactobacillus helveticus UA881 strain increases trans-epithelial electrical resistance in colon adenocarcinoma cell line C2BBe1 cells, in which MRS is the medium for culturing the Lactobacillus helveticus UA881 strain.

FIG. 2 shows that the Lactobacillus helveticus UA881 strain promotes barrier repair on lipopolysaccharide (LPS)-induced delay in wound healing, in which **p<0.01.

FIG. 3 shows the effect of the Lactobacillus helveticus UA881 strain on tight junction ZO-1 structure in C2BBe1 cells.

FIG. 4 shows that the Lactobacillus helveticus UA881 strain massively enhances macrophage glutathione (GSH) content, in which *** indicates compared with Mock, p<0.001.

FIG. 5 shows that the Lactobacillus helveticus UA881 strain has the ability to degrade purine nucleosides, in which ATCC 15009 represents Lactobacillus helveticus ATCC 15009, LH006 represents a Lactobacillus helveticus strain isolated from human breast milk, and compared with ATCC 15009, *p<0.05; **p<0.01; ***p<0.001.

FIG. 6 shows that the Lactobacillus helveticus UA881 strain has the ability to degrade uric acid, in which ATCC 15009 represents Lactobacillus helveticus ATCC 15009, LH006 represents a Lactobacillus helveticus strain isolated from human breast milk, and compared with ATCC 15009, *p<0.05; **p<0.01; ***p<0.001.

FIG. 7 shows that the Lactobacillus helveticus UA881 strain can reduce macrophage-induced inflammation, in which compared with LPS, ***p<0.001. This result implies the potential of UA881 to relieve gout arthritis.

FIG. 8 shows that the Lactobacillus helveticus UA881 strain is able to produce higher amount of folic acid comparing to other Lactobacillus helveticus strains, in which ATCC 15009 represents Lactobacillus helveticus ATCC 15009, LH006 represents a Lactobacillus helveticus strain isolated from human breast milk, and compared with ATCC 15009, *p<0.05; **p<0.01; ***p<0.001.

FIG. 9 shows that the Lactobacillus helveticus UA881 strain decreases serum triacylglyceride (TG) in the clinical trial, in which compared with day.

FIG. 10 shows that the Lactobacillus helveticus UA881 strain reduces serum cholesterol level to within the normal range in the clinical trial, in which p<0.05 compared with day 0.

FIG. 11 shows that the Lactobacillus helveticus UA881 strain reduces LDL and increases HDL in serum, in which compared with day 0.

FIG. 12 shows that the Lactobacillus helveticus UA881 strain reduces pro-inflammatory cytokine secretion.

FIG. 13 shows that the Lactobacillus helveticus UA881 strain activates antioxidant systems and prevents oxidative damage, in which compared with day 0.

FIG. 14 shows that the Lactobacillus helveticus UA881 strain significantly enhances vitamin B12 in serum.

FIG. 15 shows that the Lactobacillus helveticus UA881 strain reduces serum uric acid.

### DETAILED DESCRIPTION OF THE PREFERRED EMBODIMENT

In the following detailed description of the embodiments of the present invention, reference is made to the accompanying drawings, which are shown to illustrate the specific embodiments in which the present disclosure may be practiced. These embodiments are provided to enable those skilled in the art to practice the present disclosure. It is understood that other embodiments may be used and that changes can be made to the embodiments without departing from the scope of the present invention. The following description is therefore not to be considered as limiting the scope of the present invention.

### Definition

As used herein, the data provided represent experimental values that can vary within a range of +20%, preferably within ±10%, and most preferably within +5%.

Unless otherwise stated in the context, “a”, “the” and similar terms used in the specification (especially in the following claims) should be understood as including singular and plural forms.

The “Probiotic or Probiotic bacteria” described herein is a microorganism whose cells, mixed strains, extracts, or metabolites have a positive effect on the host. The probiotic bacteria are usually derived from the individual and are beneficial to the individual's health, and can also refer to certain microorganisms that are supplemented by external sources and may be beneficial to the individual.

The “metabolites” described herein are substances secreted by a microorganism after being metabolized. More specifically, they can be substances secreted by bacteria into the bacterial culture medium during culture.

The “metabolism disorders” described herein are characterized by abnormalities in the body's metabolic processes. These disorders encompass a wide range of conditions, including but not limited to hyperlipidemia, hypercholesterolemia, elevated uric acid levels, and gout. Metabolism disorders can affect various aspects of health, ultimately posing significant health risks.

Leaky gut syndrome primarily occurs when the intestinal epithelial barrier is compromised, allowing bacteria, viruses, or toxic substances (such as pesticides, heavy metals, etc.) to directly enter the bloodstream and lymphatic system, further compromising the body. This leads to an increase in intestinal permeability, a decrease in trans-epithelial electric resistance (TEER), and a reduction in the expression of tight junction protein ZO-1. Additionally, the ability of cells to repair wounds is diminished. Ultimately, these factors contribute to inflammation and metabolism disorders.

According to the present invention, the operating procedures and parameter conditions related to bacterial culture fall within the scope of the professional literacy and routine techniques of those skilled in the art.

The Lactobacillus helveticus UA881 strain of the present invention was deposited in the National Institute Technology and Evaluation (NITE) on Feb. 1, 2023, under accession number NITE BP-03802. The Lactobacillus helveticus UA881 strain will be publicly available upon the granting of the present application.

According to the present invention, the Lactobacillus helveticus UA881 strain (NITE BP-03802) is isolated from human breast milk. The present invention shows that the Lactobacillus helveticus UA881 strain is able to lower levels of uric acid, purine nucleosides, cholesterol, and triacylglyceride. Therefore, the present invention suggests that the Lactobacillus helveticus UA881 strain has the potential to deal with metabolic disorders.

In addition, the Lactobacillus helveticus UA881 strain has anti-inflammation property and the ability to regulate the composition of intestinal flora and enhance immunity. Moreover, folic acid is an essential micro-nutrient of humans, which is responsible for cell differentiation, hematopoiesis, and relieving metabolic disorders. The Lactobacillus helveticus UA881 strain can produce a large amount of folic acid compared to other Lactobacillus helveticus strains, providing health benefits to the host consistently.

According to the present invention, a method of relieving leaky gut syndrome and metabolic disorders using probiotic is provided. In particular, the Lactobacillus helveticus UA881 strain has the following effects, including relieving leaky gut syndrome by increasing trans-epithelial electrical resistance, wound healing and zonula occludens-1 (ZO-1) level; promoting bowel movement; reducing cholesterol, triacylglyceride (TG), LDL, and uric acid; removing gout-inducing factors; enhancing serum vitamin B12 level; and activating antioxidant systems.

According to the present invention, the Lactobacillus helveticus UA881 strain can protect the intestinal barrier by maintaining the tight junction of intestinal epithelial cells, preventing leaky gut symptoms, thereby increasing antioxidant capacity and the production of nutrient sources in the body. It also reduces inflammation and lowers cholesterol, triglycerides, low-density lipoprotein, and uric acid to eliminate the triggering factors of gout.

According to the present invention, the Lactobacillus helveticus UA881 strain is a natural aid for cardiovascular health.

The probiotic composition of the present invention can be applied to the purposes of preparing a pharmaceutical composition. The pharmaceutical composition can be a medicament, a nutritional supplement, a health food, a food product, a skin health product, an external product, or a combination thereof. The pharmaceutical composition can further comprise a pharmaceutically acceptable excipient, carrier, adjuvant, and/or food additive.

In a preferred embodiment of the present invention, the probiotic composition of the present invention is formulated in a pharmaceutically acceptable vehicle and made into a dosage form suitable for oral administration, and the pharmaceutical composition is preferably in a dosage form selected from the group consisting of: solution, suspension, powder, tablet, pill, syrup, lozenge, troche, chewing gum, capsule, and the like.

According to the present invention, the pharmaceutically acceptable vehicle can comprise one or more reagents selected from the group consisting of solvent, buffer, emulsifier, suspending agent, decomposer, disintegrating agent, dispersing agent, binding agent, excipient, stabilizing agent, chelating agent, diluent, gelling agent, preservative, wetting agent, lubricant, absorption delaying agent, liposome, and the like. The selection and quantity of these reagents fall within the scope of the professional literacy and routine techniques of those skilled in the art.

According to the present invention, the pharmaceutically acceptable vehicle comprises a solvent selected from the group consisting of water, normal saline, phosphate buffered saline (PBS), aqueous solution containing alcohol, and combinations thereof.

According to the present invention, the skin health product may further comprise an acceptable adjuvant that is widely used in skin health product manufacturing techniques. For example, the acceptable adjuvant may comprise one or more reagents selected from the group consisting of: solvent, gelling agent, active agent, preservative, antioxidant, screening agent, chelating agent, surfactant, coloring agent, thickening agent, filler, fragrance, and odor absorber. The selection and quantity of these reagents fall within the scope of the professional literacy and routine techniques of those skilled in the art.

In another preferred embodiment of the present invention, the probiotic composition of the present invention can be prepared as a food product, and is formulated with edible materials to include but not limited to: beverages, fermented foods, bakery products, health foods, nutritional supplements, and dietary supplements.

According to the present invention, the edible material is selected from the group consisting of: water, fluid milk products, milk, concentrated milk; fermented milk such as yogurt, sour milk, frozen yogurt, and lactic acid bacteria-fermented beverages; milk powder; ice cream; cream cheeses; dry cheeses; soybean milk; fermented soybean milk; vegetable-fruit juices; juices; sports drinks; confectionery; jellies; candies; infant formulas; health foods; animal feeds; Chinese herbals; and dietary supplements.

According to the present invention, the food product can be regarded as a food additive, which is added during the preparation of raw materials by conventional methods, or added in the production process of food, and formulated with any edible material into food products for human and non-human animals to eat.

Lactobacillus helveticus UA881 Sample Preparation

Lactobacillus helveticus UA881 strain was inoculated into a MRS broth (BD Difco™) at an inoculum concentration of 1% (about 1×10⁶ CFU/mL) and cultivated at 37° C. for 18-24 hours, so as to provide a Lactobacillus helveticus UA881 cultivation liquid for use in the following experiments. The Lactobacillus helveticus UA881 cultivation liquid was inoculated into a MRS broth at an inoculum concentration of 1% (about 1×10⁶ CFU/mL) and cultivated at 37° C. for 18-24 hours. After the aforementioned cultivation, the broth was sterilized at 121° C. for 15 minutes and then filtered with a 0.2 μm filter. The filtrate was collected from the filtration, and a Lactobacillus helveticus UA881 sample was thus obtained.

### Example 1

Lactobacillus helveticus UA881 Strain Increases Trans-Epithelial Electrical Resistance in C2BBe1 Cells

Epithelial barrier is usually reflected by trans epithelial electric resistance (TEER) and paracellular permeability. When the barrier is disrupted, a decrease in TEER can be observed, triggering increased cell permeability, which is the main cause of leaky gut syndrome. Inducing damage through lipopolysaccharide (LPS) stimulation, the integrity of the cell monolayer is damaged, resulting in a decrease in TEER value and an increase in permeability.

The human colon adenocarcinoma cell line C2BBe1 (purchased from BCRC 60182) was seeded onto 12-well transwell inserts (Corning, cat. #3401) at a density of 5.6×10⁵ cells per well. The cells were cultured in DMEM medium supplemented with 10% fetal bovine serum and 1% penicillin/streptomycin at 37° C. with 5% CO2 for 7 days. Epithelial resistance was measured using an Epithelial Volt/Ohm Meter (EVOM2) (World Precision Instruments, Sarasota, FL) after achieving a stable plateau phase. Treatments including Mock, LPS, and LPS combined with MRS or UA881 were applied and their effects on cell barrier integrity and permeability were assessed over time.

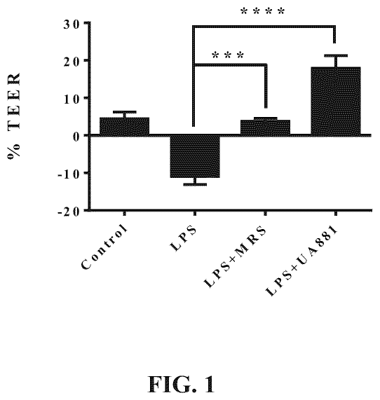

FIG. 1 shows that the Lactobacillus helveticus UA881 strain increases trans-epithelial electrical resistance in colon adenocarcinoma cell line C2BBe1 cells. As shown in FIG. 1, compared with the LPS group, the Lactobacillus helveticus UA881 strain can increase TEER by 28.9%, showing that the Lactobacillus helveticus UA881 strain can maintain cell integrity and function and avoid damage.

### Example 2

Lactobacillus helveticus UA881 Strain Promotes Barrier Repair on LPS-Induced Delay in Wound Healing

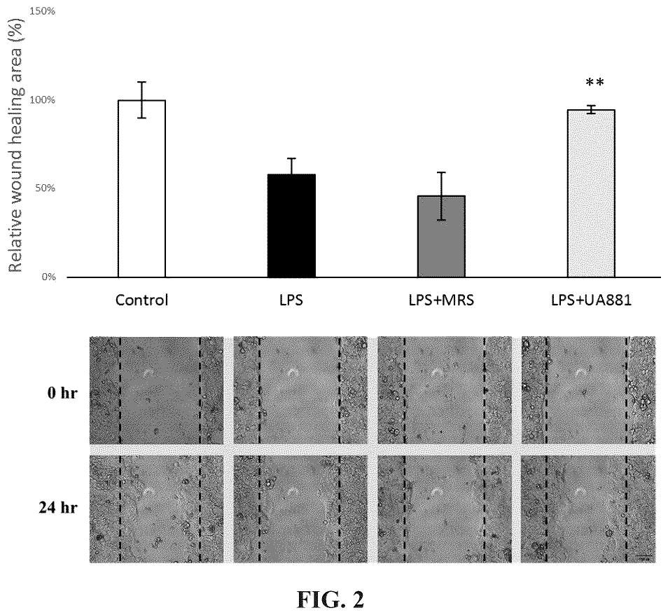

In the small intestine, repair mechanisms are initiated after barrier damage due to stimulation. The human colon adenocarcinoma cell line C2BBe1 (purchased from BCRC 60182) was seeded at a density of 5.5×10⁵ cells per well into culture inserts (purchased from IBIDI, 80209), which were adhered to six-well plates. The cells were cultured in 100 μl of DMEM medium containing 10% fetal bovine serum and 1% penicillin/streptomycin, and incubated in a constant temperature CO₂ incubator at 37° C. for 24 hours for attachment to the six-well plates. After cell attachment, the culture inserts were removed, and the cells were treated with Mock (2.0% sterile distilled water), LPS 1 μg/ml, LPS 1 μg/ml+2.0% MRS, or LPS 1 μg/ml+2.0% UA881, dissolved in DMEM medium containing 5% fetal bovine serum and 1% penicillin/streptomycin, and added at a volume of 500 μl. The cells were co-cultured with C2BBe1 cells in a 37° C. incubator for 24 hours. After the addition of the samples, the first images were captured at 0 hours, followed by recording at 24 hours, to observe the healing of cell crawling wounds. The images of cell crawling were then quantified using Image J software. Finally, the quantified values at 24 hours were compared with those at 0 hours to determine the ability of cell crawling to promote wound healing. FIG. 2 shows that the Lactobacillus helveticus UA881 strain promotes barrier repair on LPS-induced delay in wound healing, in which **p<0.01. As shown in FIG. 2, compared with the LPS group, the Lactobacillus helveticus UA881 strain can improve the repair ability by 37%.

### Example 3

Effect of Lactobacillus helveticus UA881 Strain on Tight Junction ZO-1 Structure in C2BBe1 Cells

Zonula occludens-1 (ZO-1) is a protein related to tight junctions that participates in and regulates the barrier function of cells. It is often used as an indicator to observe the barrier function and permeability function of tight junctions in various tissues. Lipopolysaccharide (LPS) is used to induce Caco-2 cell monolayer barrier damage model and reduce the expression of ZO-1 protein. The human colon adenocarcinoma cell line C2BBe1 (purchased from BCRC 60182) was seeded at a density of 1.5×10⁶ cells per well onto 21 mm*21 mm glass slides placed in six-well plates, with 2000 μl of DMEM medium containing 10% fetal bovine serum and 1% penicillin/streptomycin. After 24 hours of incubation at 37° C. with 5% CO₂, cells were uniformly attached to both the plates and slides. Following cell attachment, treatments including Mock (1.0% sterile distilled water), LPS 100 μg/ml, LPS 100 μg/ml+1.0% MRS, and LPS 100 μg/ml+1.0% UA881, dissolved in DMEM medium with 5% fetal bovine serum and 1% penicillin/streptomycin, were added at 500 μl per well. Cells were co-cultured for 24 hours at 37° C. Subsequently, slides were washed with PBS, fixed with 4% PFA, permeabilized with Triton-X100, and stained with Rabbit anti ZO-1 antibody (iReal; IR56-184) followed by Alexa Fluor 488 anti-rabbit IgG. After staining with Hoechst dye, slides were sealed and dried. Results were recorded using a fluorescence microscope. FIG. 3 shows the effect of the Lactobacillus helveticus UA881 strain on tight junction ZO-1 structure in C2BBe1 cells. As shown in FIG. 3, the Lactobacillus helveticus UA881 strain can increase the expression of ZO-1 protein and regulate the expression of tight junction proteins in intestinal epithelial cells, thereby protecting the intestinal barrier.

### Example 4

Lactobacillus helveticus UA881 Strain Massively Enhances Macrophage GSH Content

Glutathione (GSH) is a powerful antioxidant in human cells, which is indispensable for preventing ROS damage. GSH also helps detoxification of drugs and pollutants in the liver, DNA synthesis or repair, and regulation of the immune system. Intracellular GSH content has a strong correlation with anti-aging and overall health.

GSH is composed of glutamic acid, cysteine and glycine, of which the thiol group of cysteine is the main functional group. As an antioxidant in animal cells, it can protect DNA from oxidation. The assay uses the glutathione assay kit (Cayman #703002), which uses glutathione reductase in the enzyme cycle redox to quantify the GSH content. The thiol group of GSH would react with DTNB to produce yellow TNB, and the absorbance is measured at 405-414 nm.

RAW264.7 macrophages were cultured in a 10-cm dish (2×10⁵ cell), and the samples to be tested were cultured for 24 hours. The culture medium was removed, rinsed with PBS, and the cells were scraped from the 10-cm dish with a cell scraper. Centrifugation was performed at 1500×g for 10 minutes at 4° C., the supernatant was removed and the pellet was re-suspended in 50 mM phosphate buffer (pH7, containing 1 mM EDTA). After homogenization, centrifugation was performed at 10000×g for 15 minutes at 4° C., an equal volume of MPA reagent (5 g metaphosphoric acid+50 mL DDW) was added to the supernatant, followed by shaking for 5 minutes. Centrifugation was performed at 3000×g for 5 minutes, the supernatant was aspirated, and 50 μl of TEAM reagent (4M triethanolamine) was added per ml, followed by shaking. 50 μl of each sample was added to a 96-well plate, 150 μl of Assay cocktail reagents were add in sequence, shaking for 25 minutes, and the absorbance was measured at 405-414 nm.

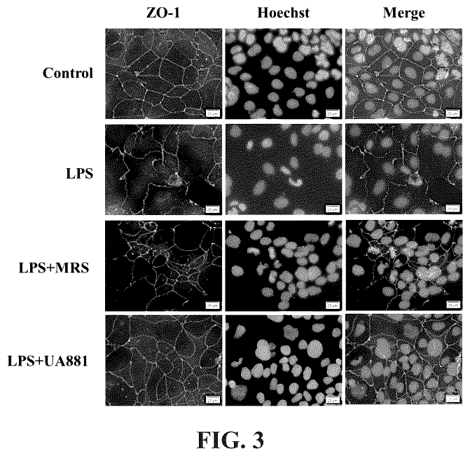

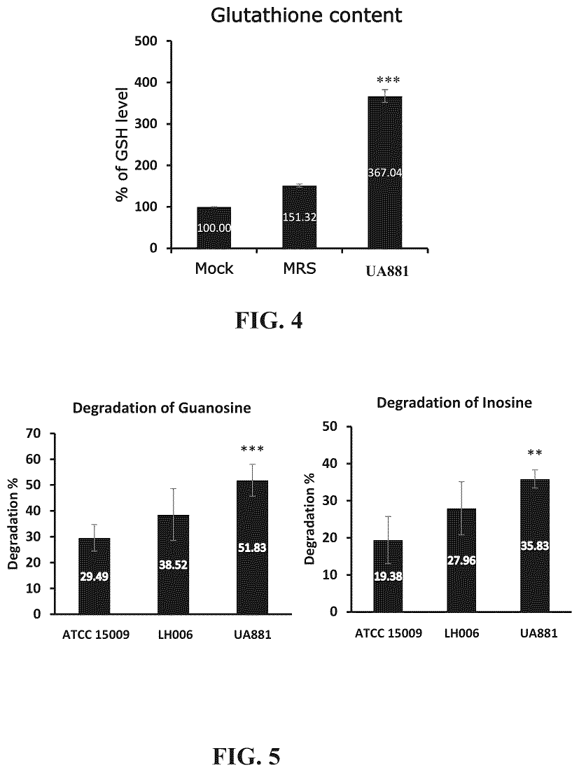

FIG. 4 shows that the Lactobacillus helveticus UA881 strain massively enhances macrophage GSH content, in which *** indicates compared with Mock, p<0.001. As shown in FIG. 4, the Lactobacillus helveticus UA881 strain can significantly stimulate GSH biosynthesis in immune cells. GSH content is 3.67-fold higher in the Lactobacillus helveticus UA881 strain treating group.

### Example 5

Lactobacillus helveticus UA881 Strain has Ability to Degrade Purine Nucleosides

The assay for UA881 purine nucleosides degradation capability involves the following steps: (a) Dissolve 1 mM Guanosine and 1 mM Inosine in 100 mM K₃PO₄ solution, adjusted to pH 7. (b) Wash 3.2 ml of appropriately activated UA881 with 0.85% NaCl, then resuspend in 1.2 ml of 1 mM guanosine/inosine aqueous solution. (c) Incubate at 37° C. with shaking at 160 rpm for 2 hours. (d) Centrifuge at 8000 rpm for 10 minutes. (e) Take out 1 ml of supernatant and mix with 0.111 ml of perchloric acid to stop the reaction. (f) Filter through a 0.22 μm PVDF membrane and analyze Guanosine and Inosine content using HPLC. (g) HPLC conditions: Column: C18, Wavelength: 254 nm, Temperature: 30° C., Flow rate: 1 ml/min, Injection volume: 0.02 ml, Mobile phase: 0.1 mM NaClO₄+0.187 M H₃PO₄. FIG. 5 shows that the Lactobacillus helveticus UA881 strain has the ability to degrade purine nucleosides, in which ATCC 15009 represents Lactobacillus helveticus ATCC 15009, LH006 represents a Lactobacillus helveticus strain isolated from human breast milk, and compared with ATCC 15009, *p<0.05; **p<0.01; ***p<0.001.

As shown in FIG. 5, through HPLC analysis, the Lactobacillus helveticus UA881 strain can degrade guanosine and inosine efficiently within 2 hours. The degradation rates are significantly higher than the type strain.

### Example 6

Lactobacillus helveticus UA881 Strain has Ability to Degrade Uric Acid

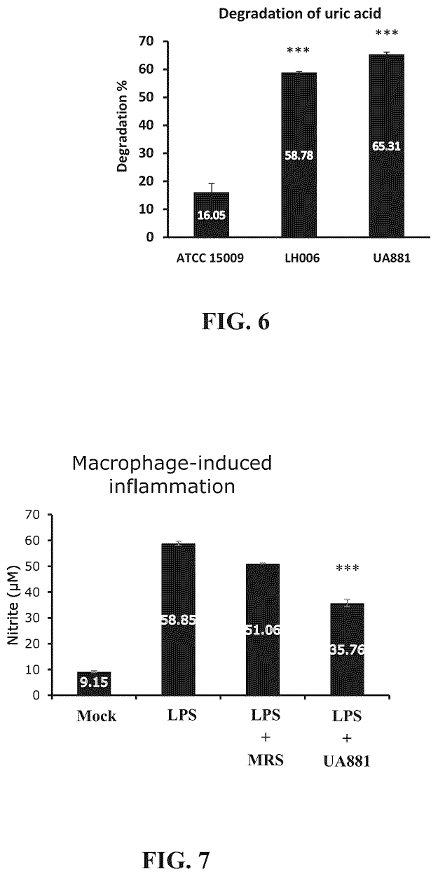

The method for assessing UA881's uric acid degradation ability involves several steps. Firstly, 100 mM uric acid is dissolved in a 100 mM K₃PO₄ solution at pH 7. Subsequently, 3.2 ml of appropriately activated UA881 is washed with 0.85% NaCl and then resuspended in 1.2 ml of the uric acid solution. The mixture is then incubated at 37° C. with shaking at 160 rpm for 2 hours, followed by centrifugation at 8000 rpm for 10 minutes. After centrifugation, 1 ml of supernatant is taken and mixed with 0.111 ml of perchloric acid to halt the reaction. The resulting solution is filtered through a 0.22 μm PVDF membrane and the uric acid content is analyzed using HPLC under the following conditions: C18 column, 254 nm wavelength, 30° C. temperature, 1 ml/min flow rate, 0.02 ml injection volume, and a mobile phase consisting of 0.1 mM NaClO₄+0.187 M H₃PO₄. FIG. 6 shows that the Lactobacillus helveticus UA881 strain has the ability to degrade uric acid, in which ATCC 15009 represents Lactobacillus helveticus ATCC 15009, LH006 represents a Lactobacillus helveticus strain isolated from human breast milk, and compared with ATCC 15009, *p<0.05; **p<0.01; ***p<0.001. As shown in FIG. 6, the Lactobacillus helveticus UA881 strain has uricase activity, having the ability to degrade uric acid. The degradation rate is 49% higher than the type strain. In conclusion, the Lactobacillus helveticus UA881 strain may improve hyperuricemia from the beginning to the end of the uric acid degradation pathway in the intestinal tract.

### Example 7

Lactobacillus helveticus UA881 Strain can Relieve Gout Arthritis

Gout patients suffer from inflammatory arthritis, which can lead to joint damage and extreme pain. Macrophages play important roles in inducing inflammation by secreting nitric oxide and cytokines.

The Griess Reagent Kit ab234044 uses a classic protocol for the estimation of nitrite in biological samples. During the assay, RAW264.7 were cultured in a 6-cm dish (1.5×10⁶ cell). After adding the test substance and culturing for 24 hours, the cell supernatants were collected after co-culturing with LPS (0.1 μg/mL) for 24 hours. 100 μl of the supernatant was added into a 96-well plate, and 10 μl of Griess reagent R1 and 10 μl of Griess reagent R2 were added. After reacting at room temperature for 10 minutes, an ELISA reader was used to measure the absorbance at a wavelength of 540 nm, and the absorbance was substituted into the standard curve to calculate the nitrite content.

FIG. 7 shows that the Lactobacillus helveticus UA881 strain has the potential to relieve gout arthritis, in which compared with LPS, ***p<0.001. As shown in FIG. 7, the Lactobacillus helveticus UA881 strain significantly inhibits macrophage-induced inflammation, showing anti-inflammation ability.

### Example 8

Lactobacillus helveticus UA881 Strain is Able to Produce Higher Amount of Folic Acid Comparing to Other Lactobacillus helveticus Strains

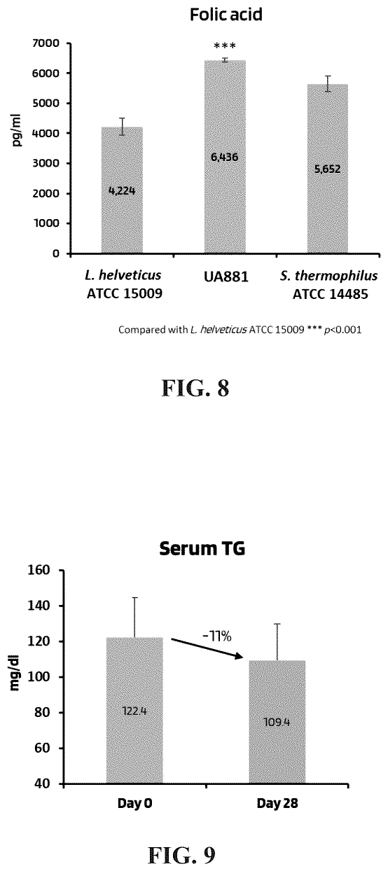

As shown in FIG. 8, Lactobacillus helveticus UA881 possesses remarkable capability in folic acid production, compared to other strains of Lactobacillus helveticus and Streptococcus thermophilus, exhibited a significantly higher folic acid level. Specifically, ATCC 15009 represents Lactobacillus helveticus ATCC 15009, while ATCC 14485 denotes a commonly-used Streptococcus thermophilus strain for folic acid production in the industry. The results were compared with those of ATCC 15009, with significance levels denoted as follows: *p<0.05; **p<0.01; ***p<0.001.

As shown in FIG. 8, the Lactobacillus helveticus UA881 strain is able to produce higher amount of folic acid comparing to other Lactobacillus helveticus strains.

Effect of Lactobacillus helveticus UA881 Strain of Present Invention on Promotes Cardiovascular Health by Regulating Blood Lipids and Cholesterol, Generating Micro-Nutrients, and Activating Antioxidant Systems

The process of the human trial is shown in Table 1.

TABLE 1

| | | Subject | | Sampling |
|---|---|---|---|---|
| Dosage: | Subject: | criteria: | Duration: | time point: |
| 1-10 billion | 5 persons | Working people | 28 days | Day 0 |
| CFU/day | | aged from 20 to 65 | | Day 28 |

Biomarkers (all are entrusted to medical examination institutes):
Serum Triacylglyceride
Serum Cholesterol
Serum LDL-C, HDL-C
Inflammation Cytokine (IL-8)
Antioxidant activity (SOD and Catalase)
Oxidative Damage Marker (MDA)
Vitamin B12
Serum Uric acid

### Example 9

Lactobacillus helveticus UA881 Strain Decreases Serum Triacylglyceride (TG) in Clinical Trial

Serum TG is the major risk factor for heart attack and stroke. FIG. 9 shows that the Lactobacillus helveticus UA881 strain decreases serum triacylglyceride (TG) in the clinical trial. As shown in FIG. 9, compared with day 0, the Lactobacillus helveticus UA881 strain lowers 11% TG in serum after 4 weeks of consumption, having the potential to alleviate hyperlipidemia.

### Example 10

Lactobacillus helveticus UA881 Strain Reduces Serum Cholesterol Level to within Normal Range in Clinical Trial

Excess cholesterol is the risk factor for cardiovascular disease, which remains 1^{st} leading cause of mortality worldwide. The recommended cholesterol level is below 200 mg/dl.

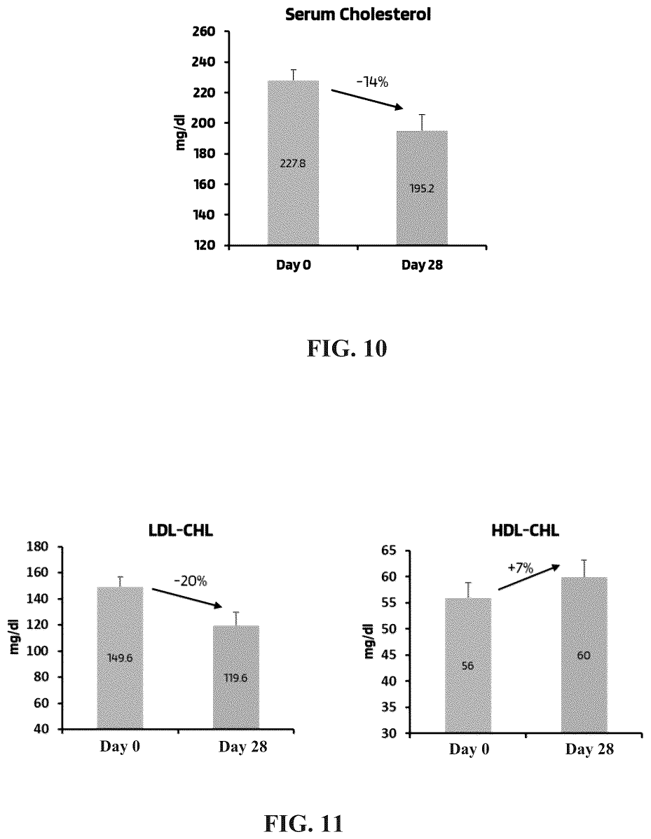

FIG. 10 shows that the Lactobacillus helveticus UA881 strain reduces serum cholesterol level to within the normal range in the clinical trial, in which p<0.05 compared with day 0. As shown in FIG. 10, consumption of the Lactobacillus helveticus UA881 strain significantly decreases serum cholesterol level and even restores it to the normal range.

### Example 11

Lactobacillus helveticus UA881 Strain Reduces LDL and Increases HDL in Serum

Low-density lipoprotein (LDL)-cholesterol is often referred to as “bad cholesterol”, leading to the buildup of plaque in the arteries. High-density lipoprotein (HDL)-cholesterol represents “good cholesterol”, and helps remove LDL cholesterol from the bloodstream.

FIG. 11 shows that the Lactobacillus helveticus UA881 strain reduces LDL and increases HDL in serum, in which compared with day 0.

As shown in FIG. 11, the Lactobacillus helveticus UA881 strain helps participants reach the normal range after 4 weeks of consumption. Taking the Lactobacillus helveticus UA881 strain for 28 days can reduce 31.2% risk of stroke.

### Example 12

Lactobacillus helveticus UA881 Strain Reduces Pro-Inflammatory Cytokine Secretion

IL-8 is a pivotal cytokine in cardiovascular disease development. Elevated levels of IL-8 are associated with an increased risk of future cardiovascular disease.

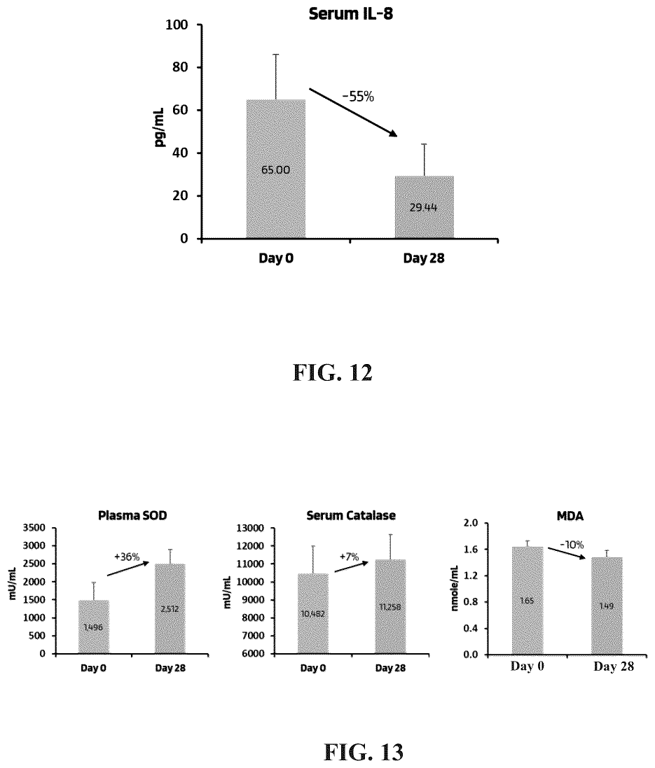

FIG. 12 shows that the Lactobacillus helveticus UA881 strain reduces pro-inflammatory cytokine secretion. As shown in FIG. 12, compared with day 0, the Lactobacillus helveticus UA881 strain reduces 55% serum IL-8, suggesting its efficacy on promoting cardiovascular health.

### Example 13

Lactobacillus helveticus UA881 Strain Activates Antioxidant Systems and Prevents Oxidative Damage

Oxidant stress has been linked to the pathogenesis of cardiovascular disease. Antioxidants serve as a preventive measure to reduce the risk of heart disease.

FIG. 13 shows that the Lactobacillus helveticus UA881 strain activates antioxidant systems and prevents oxidative damage. As shown in FIG. 13, compared with day 0, the Lactobacillus helveticus UA881 strain increases 36% SOD and 7% catalase activity, and further alleviates oxidative damage.

### Example 14

Lactobacillus helveticus UA881 Strain Significantly Enhances Vitamin B12 in Serum

B12 is an essential vitamin for red blood cell production, DNA synthesis, and neurotransmitter synthesis. Lacking of B12 leads to cardiovascular diseases, anemia, neurological symptoms, and even Alzheimer's disease.

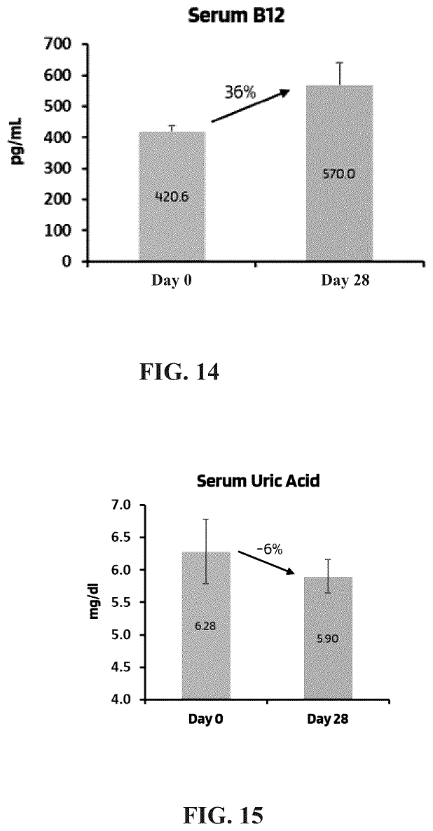

FIG. 14 shows that the Lactobacillus helveticus UA881 strain significantly enhances vitamin B12 in serum.

As shown in FIG. 14, the Lactobacillus helveticus UA881 strain acts as a nature B12 booster. Serum B12 significantly increased by 36% after 4 weeks of the Lactobacillus helveticus UA881 strain supplementation.

### Example 15

Lactobacillus helveticus UA881 Strain Reduces Serum Uric Acid

FIG. 15 shows that the Lactobacillus helveticus UA881 strain reduces serum uric acid. As shown in FIG. 15, the Lactobacillus helveticus UA881 strain reduces 6% of serum uric acid after 28 days of consumption.

In summary, the Lactobacillus helveticus UA881 strain of the present invention has the effects on producing folic acid in the human intestine, degrading uric acid, purine nucleosides, cholesterol, relieving leaky gut syndrome and metabolic disorders through the experiments illustrated in the above mentioned examples.

Although the present invention has been described with reference to the preferred embodiments, it will be apparent to those skilled in the art that a variety of modifications and changes in form and detail may be made without departing from the scope of the present invention defined by the appended claims.

What is claimed is:

1. A probiotic composition, comprising a Lactobacillus helveticus UA881 strain and/or its metabolites, wherein the Lactobacillus helveticus UA881 strain is deposited in National Institute Technology and Evaluation (NITE) under an accession number NITE BP-03802.

2. The probiotic composition according to claim 1, wherein the Lactobacillus helveticus UA881 strain is viable, inactive or its metabolites.

3. A method for intestinal permeability and metabolic disorders by using the probiotic composition according to claim 1.

4. The method according to claim 3, wherein the probiotic composition is a medicament, a nutritional supplement, a health food, a food product, an external product, or a combination thereof.

5. A method for relieving leaky gut syndrome, comprising administering to a subject in need thereof a pharmaceutical composition comprising an effective amount of the probiotic composition according to claim 1.

6. The method according to claim 5, wherein the probiotic composition increases trans-epithelial electrical resistance in colon adenocarcinoma cells.

7. The method according to claim 5, wherein the probiotic composition promotes barrier repair on lipopolysaccharide (LPS)-induced delay in wound healing.

8. The method according to claim 5, wherein the probiotic composition increases expression of zonula occludens-1 (ZO-1) protein and regulates expression of tight junction proteins in intestinal epithelial cells, thereby protecting intestinal barrier.

9. The method according to claim 5, wherein the pharmaceutical composition is a medicament, a nutritional supplement, a health food, a food product, a skin health product, an external product, or a combination thereof.

10. A method for relieving metabolic disorders, comprising administering to a subject in need thereof a pharmaceutical composition comprising an effective amount of the probiotic composition according to claim 1.

11. The method according to claim 10, wherein the probiotic composition enhances macrophage glutathione (GSH) content.

12. The method according to claim 10, wherein the probiotic composition has ability to degrade purine nucleosides.

13. The method according to claim 10, wherein the probiotic composition has ability to degrade uric acid.

14. The method according to claim 10, wherein the probiotic composition decreases serum triacylglyceride (TG).

15. The method according to claim 10, wherein the probiotic composition reduces serum cholesterol level to within normal range.

16. The method according to claim 10, wherein the probiotic composition reduces low-density lipoprotein (LDL) and increases high-density lipoprotein (HDL) in serum.

17. The method according to claim 10, wherein the probiotic composition reduces pro-inflammatory cytokine secretion.

18. The method according to claim 10, wherein the probiotic composition activates antioxidant systems and prevents oxidative damage.

19. The method according to claim 10, wherein the probiotic composition enhances vitamin B12 and folic acid.

20. A method for relieving gout arthritis, comprising administering to a subject in need thereof a pharmaceutical composition comprising an effective amount of the probiotic composition according to claim 1.
